(19) United States
(12) Patent Application Publication
(10) Pub. No.: US 2009/0104236 A1
(43) Pub. Date: Apr. 23, 2009

(54) PHARMACEUTICAL SOLID HYBRIDS

(75) Inventor: Emadeldin Hassan, Towson, MD (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Pharmaceutics International, Inc., Hunt Valley, MD (US)

(21) Appl. No.: 11/874,708

(22) Filed: Oct. 18, 2007

(51) Int. Cl. A61K 9/00

(52) U.S. Cl. 424/400

(57) ABSTRACT

The invention provides solid pharmaceutical hybrids that are useful in the treatment or diagnosis of diseases, as well as methods of producing the solid pharmaceutical hybrids.

### BACKGROUND OF THE INVENTION

The major approach in drug discovery is focused on selecting one-best therapeutic agent after screening thousands of possible molecules, which are suggested by combinatorial techniques. Similarly, the one-best candidate paradigm is applied to the pre-formulation discovery process, to identify one drug chemical form, and/or one drug physical form for further formulation development and optimization. The tedious pre-formulation screening of drug chemical and physical forms can be expedited using advanced throughput techniques especially for salt and polymorph screening (see, e.g., U.S. Pat. No. 6,939,515, U.S. Pat. No. 6,750,064, and U.S. Pat. No. 6,808,934).

Regardless of expediting the screening process, there is always a possibility for one selected drug form to show some limitation with time. In fact, inappropriate selection of drug forms has repeatedly required extensive additional efforts to identify other alternatives or to introduce special formulation techniques to overcome drawbacks of the already existing forms.

U.S. Pat. No. 6,245,917 (which describes a single crystal form of phenytoin sodium hydrate), U.S. Pat. No. 6,696,600 (which describes non-hygroscopic salts of L-dopa ethyl ester) and U.S. Pat. No. 6,160,020 (which describes discrete salts of acetaminophen) illustrate a few recent examples of alternative forms of drugs.

Examples of formulation approaches to improve drug form performance include the use of surfactants and ionizing agents (see, e.g., U.S. Pat. No. 6,383,471), complexation (see, e.g., U.S. Pat. No. 5,997,856), solid dispersion (see, e.g., U.S. Pat. No. 5,776,495), or co-crystals (see, e.g., U.S. Pat. No. 7,078,526). Formulation approaches usually require sophisticated techniques and/or special equipment. In addition, active agents are subjected to different degrees of dilutions that can be problematic with high drug loading.

Accordingly, there is a need for alternatives to the classic one-best drug form that reduce the risk of drug form screening failures, as well as the time and efforts involved in the traditional pre-formulation process.

### BRIEF SUMMARY OF THE INVENTION

The invention provides a composition comprising a solid pharmaceutical hybrid, wherein the solid pharmaceutical hybrid comprises two or more forms of an agent, wherein the agent is a pharmacologically active agent, a therapeutic agent, or a diagnostic agent, and wherein the solid pharmaceutical hybrid exhibits at least one superior property relative to one of the forms of the agent.

The invention also provides a method of producing a solid pharmaceutical hybrid comprising two or more forms of an agent, comprising mixing a form of the agent with one or more different forms of the agent, wherein the agent is a pharmacologically active agent, a therapeutic agent, or a diagnostic agent, and wherein the solid pharmaceutical hybrid exhibits at least one superior property relative to one of the forms of the agent.

The invention also provides a method of producing a solid pharmaceutical hybrid comprising two or more forms of an agent, wherein the method comprises actively mixing one or more forms of an agent with one or more hybrid forming agents in a medium, such that the solid pharmaceutical hybrid is produced.

### DETAILED DESCRIPTION OF THE INVENTION

The invention provides a composition comprising a solid pharmaceutical hybrid and a method of manufacturing the solid pharmaceutical hybrid. Solid pharmaceutical hybrids are mixtures of two or more different forms of an agent, wherein the agent is a pharmacologically active agent, therapeutic agent, or diagnostic agent.

The agent for use in the solid pharmaceutical hybrid can be any suitable agent, including, but not limited to, antihypertensives, antivirals, lipid lowering agents, antipsychotics, anticancer agents, anti-inflammatories (e.g., non-steroidal anti-inflammatory drugs (NSAIDs), such as ibuprofen, naproxen, and acetaminophen), antiasthmatics, antidiarrheal, antidepressants, antibiotics (e.g., tetracycline and erythromycin), antitussives, antihistamines, decongestants, laxatives, antacids, anti-cholesterolemics, antiarrhythmics, antipyretics, analgesics, appetite suppressants, expectorants, anti-anxiety agents, anti-ulcer agents, coronary dilators, cerebral dilators, peripheral vasodilators, anti-infectives, psycho-tropics, antimanics, stimulants, gastrointestinal agents, sedatives, antidiarrheal preparations, anti-anginal drugs, vasodialators, vasoconstrictors, migraine treatments, tranquilizers, anti-psychotics, anticoagulants, antithromobotic drugs, hypnotics, antiemetics, anti-nauseants, anti-convulsants, neuromuscular drugs, hyper- and hypoglycemic agents, thyroid and antithyroid preparations, diuretics, antispasmodics, uterine relaxants, antiobesity drugs, anabolic drugs, erythropoietic drugs, antiasthmatics, cough suppressants, mucolytics, H₂-antagonists, and anti-uricemic drugs. In particular, the agent can be ibuprofen, naproxen, or tetracycline. The different forms of the agent can include chemical and physical forms of the agent.

In one embodiment, the two or more forms of the agent are chemical forms of the agent. For example, the solid hybrid pharmaceutical hybrid can comprise an ionizable form (i.e., salt) and a non-ionizable form (i.e., free form of an agent, such as an acid or base) of the agent. Alternatively or additionally, the solid pharmaceutical hybrid can comprise at least two different polymorphs of the agent. The solid pharmaceutical hybrid also can comprise at least two forms of the agent selected from the group consisting of the agent itself, a salt of the agent, an ester of the agent, a pro-drug of an agent, and an active metabolite of the agent. Each form can exist as a free molecule or in the form of a complex.

In another embodiment, the two or more forms of the agent are physical forms of the agent. For example, the solid pharmaceutical hybrid can comprise an anhydrous form and a hydrous form of the agent. Alternatively or additionally, the solid pharmaceutical hybrid can comprise an amorphous and a crystalline form of the agent. The solid pharmaceutical hybrid also can comprise at least two different particle size populations of the agent, at least two crystalline forms of the agent, at least two different solvates (e.g., hydrates) of the agent, at least two particles of differing geometric shapes (i.e., at least two populations of particles of differing geometric shapes), at least two particles of differing bulk densities, and/or at least two particles of differing flowability.

The solid pharmaceutical hybrid of the invention exhibits at least one superior property relative to one of the forms of the agent (i.e., one of the individual components of the solid pharmaceutical hybrid). The superior property can be, for example, increased solubility, increased stability, increased bioavailability, reduced side effects, and increased activity of the agent. The change in properties exhibited in the solid pharmaceutical hybrid relative to one of the forms of the agent can be any suitable change. For example, the solid pharmaceutical hybrid of the invention can exhibit at least about 1% (e.g., at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%) increased solubility, increased stability, increased bioavailability, increased activity, and/or reduced side effects relative to one of the forms of the agent. These properties can be measured by any suitable methods known in the art.

In particular, solid pharmaceutical hybrids reduce the risk of drug form screening failures and the time and efforts involved in the traditional pre-formulation process, which are steps that are typically necessary when utilizing a one-best drug system. By combining at least two chemical and/or physical forms of the agent, the solid pharmaceutical hybrid of the invention overcomes the chemical and/or physical limitations of the one-best drug form (i.e., a single form of the agent).

The components of a solid pharmaceutical hybrid (i.e., the different forms of the agent) are selected and manufactured into the hybrid to reduce the drug's physical and/or chemical limitations, improve drug properties, and/or optimize drug performance. Furthermore, the manufacturing of the solid pharmaceutical hybrid of the invention does not require tedious purification steps usually required in preparing a single drug form.

Chemical limitations of drugs include transformation to inactive material via oxidation, reduction, hydrolysis, polymerization, or light or metal catalysis. Improving chemical properties includes stabilizing chemical integrity of the drug molecule by preventing or reducing the rate of chemical degradation due to oxidation, reduction, hydrolysis, polymerization, or light or heavy metal catalysis.

Physical limitations of drugs include poor solubility in water or in physiological fluids or pharmaceutically acceptable aqueous or non-aqueous vehicles, solvents, or mixtures thereof. Physical limitations of drugs also include poor compressibility, poor powder flow, low bulk or tapped density, too low melting points that negatively impact handling or formulation of solid dosage forms, unstable or meta-stable crystal forms that transform into highly structured crystals with poor dissolution rates and subsequent poor bioavailability, high affinity to moisture or water uptake which may lead to deliquescence, undesirable crystal shapes such as needle or rod-like shapes, and physical instability. Physical instability includes undesirable changes in physical properties with time, such as melting point, color, and crystal form. Improving of drug physical properties includes increasing or controlling dissolution rate or drug release, increasing drug flowability and compressibility, stabilizing drug color or crystal shape (habit) or form (polymorph), reducing water uptake or hygroscopic behavior, and/or modifying the drug partition coefficient between lipophilic and hydrophilic media to allow the transport of the drug to diseased tissues.

Drug flowability of powder is typically measured by the angle of repose. The flowability permits the filling of the solid pharmaceutical hybrid into a finished dosage form, such as tablets or capsules (e.g., hard gelatin capsules). Generally, the solid pharmaceutical hybrid has a lower angle of repose and, thus, improved flowability as compared to a one-drug composition (e.g., less than about 30°, less than about 28°, less than about 26°, less than about 25°, less than about 22°, or less than about 20°).

Optimizing drug performance can be achieved by reducing side effects, increasing the therapeutic effect of the agent, and improving patient compliance. Side effects to be reduced include irritation of tissues or mucous membranes and impairment of enzymatic activities or normal function of vital body organs, such as stomach, intestine, liver, kidney, heart, lungs, and/or brain.

Increasing the therapeutic effect can be achieved by increasing the tolerance to higher drug dose, avoiding fluctuating blood drug concentrations out of the therapeutic window, and/or increasing the extend rate of drug absorption or bioavailability, improving drug tissue distribution, improving protein or receptor binding, improving polymorph crystallinity, and/or improving amorphous partition coefficient susceptibility to chelating or complexation with heavy metals.

In one embodiment, the solid pharmaceutical hybrid extends the release of the drug due to the at least two different forms of the agent. For example, one form of the agent present in the hybrid can have an immediate release and another form of the agent present in the hybrid can have a delayed release. In particular, when the hybrid comprises a free acid of the agent and a salt (e.g., sodium salt) of the agent, the salt has an immediate release and the free acid has a delayed release. When the hybrid comprises a water-soluble form of the agent and a water-insoluble form, the water-soluble form has an immediate release and the water-insoluble form has a delayed release. When the hybrid comprises at least two different salts of the agent, one salt (e.g., a hydrochloric salt) has an immediate release, while another salt (e.g., maleate salt) has a delayed release.

The solid pharmaceutical hybrid of the invention can have any suitable dissolution profile. In one embodiment, the solid pharmaceutical hybrid is formulated for immediate release and at least about 85% (e.g., about 87%, about 90%, about 92%, about 95%, about 97%, about 98%, about 99%, or ranges thereof) of the agent dissolves after about 30 minutes in any suitable dissolution medium. The solid pharmaceutical hybrid may optionally be formulated in any suitable dosage form to achieve this dissolution rate, for example, in a soft gelatin capsule. The dissolution rate of the solid pharmaceutical hybrid may be measured by any suitable method in any suitable medium. For example, a USP dissolution apparatus type II may be used. The dissolution medium may be, for example, purified water, gastric fluid or a potassium phosphate buffer having a pH of 7.2 containing 1% pancreatin (supplied by Sigma-Aldrich).

In an embodiment of the invention, the solid pharmaceutical hybrid is more soluble than the one-best form of the agent in any suitable dissolution medium, for example, purified water. Preferably, the solid pharmaceutical hybrid is about 2 to about 10 (e.g., about 3, about 4, about 5, about 6, about 7, about 8, about 9, or ranges thereof) times more soluble than the one-best form of the agent. More preferably, the solid pharmaceutical hybrid is 3 to 5 times more soluble than the one-best form of the agent.

Solid pharmaceutical hybrids are drug concentrates that can be prepared from one or more forms of an active ingredient using one or more hybrid forming agents in a suitable hybrid medium to yield a hybrid of two or more forms of the active ingredient. The hybrids of this invention are distinguished from traditional formulations in that they are essentially made in absence of pharmaceutical fillers that can negatively affect the hybrid formation.

To form the solid pharmaceutical hybrid, a hybrid forming agent, such as physical energy (e.g., mechanical or thermal energies) or a chemical reagent (e.g., pharmaceutically acceptable ionizing or de-ionizing agents, ester-forming agents, and chelating or complexing agents) can be used.

The solid pharmaceutical hybrid can be formed in essentially fluid media. Fluid media include air, carbon dioxide, nitrogen, vapors of acceptable solvents or other pharmaceutically acceptable gases, liquids such as water, or water-miscible non-aqueous vehicles. Liquid media are generally preferred as they allow improved surface reactions and displace air from hydrophilic surfaces. The presence of liquid media is especially preferred at amounts that facilitate wet mass formation to achieve optimum mixing and/or desirable production of drug forms such as solvates. Fluid media can also be a combination of a gas and a liquid as in case of fluid bed processing.

The solid pharmaceutical hybrids also can be formed in a solid state when compression is used. Compression can be achieved by powder roller compactors or using a tablet press. Compression at forces causing powder or crystal shape deformation is preferred to form solid pharmaceutical hybrids in solid media.

In one embodiment, the solid pharmaceutical hybrid is formed by an ionizable component and ionizing agent. In another embodiment, the solid pharmaceutical hybrid is formed by an ionized molecule and a de-ionizing agent.

The solid pharmaceutical hybrid can be prepared by any suitable method. For example, the solid pharmaceutical hybrid can be made by an active mixing process of its separately-prepared components or by generating at least one component from another component (e.g., generating an acid form of the agent by reacting a salt of the agent with an acid) while active mixing is applied.

Active mixing can be accomplished by generating new surfaces of at least one of the hybrid components and reacting the at least one hybrid component with other hybrid components to produce a desirable physical or chemical property. Since active mixing involves the generation of new surfaces, such surfaces will be exposed to the hybrid-forming agent, fluid, and/or other forms of the agent as soon as the forms are generated. The exposure of the new surface to these materials allows the exposed surfaces to acquire a primary adsorbed molecular layer from at least one of the materials involved in making the hybrid (e.g., the agent and fluid), which improves the physical or chemical characteristics of the hybrid's components.

For example, when a poorly water-soluble acid form of an agent is actively mixed with an alkali, a salt form is generated of the active agent, as well as free water molecules. The generated salt (a first hybrid component) and water can deposit on the remaining acid form, which transforms the hydrophobic surface of the acid (a second hybrid component) to a more hydrophilic surface that is easily wettable with water or aqueous physiological fluids. In this way, enhanced dissolution rates can be achieved.

As another example, when an ionized (salt) form of an active ingredient (a first hybrid component) is actively mixed with a de-ionizing hybrid forming agent, a non-ionized form of the active agent (a second hybrid component) is generated along with a more ionized by-product (a third hybrid component), which can improve the physical properties of at least one of the forms of the hybrid active ingredient. For instance, when naproxen sodium is actively mixed with lactic acid, it results in the formation of naproxen free acid and sodium lactate. Sodium lactate is highly hydrophilic, which makes the generated naproxen acid much more wettable than pure naproxen powder.

Similarly, when generating a hybrid by actively mixing (e.g., grinding) one polymorph, a hybrid of two polymorphic structures will be formed with unique surface properties superior to at least one of the polymorphs. A polymorphic hybrid can possess improved physical properties, such as fast dissolution rate, good compressibility, and/or a structurally more stable solid with a slow rate of polymorphic transition.

Active mixing can be performed with standard equipment normally used in dry or wet milling, dry or wet granulation, or spray drying. The mixing can be achieved using any suitable mixing apparatus, such as by use of common blenders known in the art. For example, suitable mixing apparatus include air-dependent mixers, such as fluid bed driers/granulators (e.g. Glatt fluid bed granulators or Wurster coaters), and mechanical enforcement mixers, such as high speed-high shear mixers (e.g., a T. K. Fielder) or low speed planetary or ribbon blenders or extruders.

The solid pharmaceutical hybrids can comprise at least two forms of the agent (i.e., hybrid components). Preferably, the solid pharmaceutical hybrid comprises about 2-20 components (e.g., 3 components, 4 components, 5 components, 6 components, 7 components, 8 components, 9 components, 10 components, 11 components, 12 components, 13 components, 14 components, 15 components, 16 components, 17 components, 18 components, 19 components, or ranges thereof), and more preferably about 2-10 components.

Each component can comprise at least about 1% of the total hybrid weight, and preferably about 5% to about 95% of the total hybrid weight (e.g., about 10%, about 15%, about 20%, about 25%, about 30%, about 35%, about 40%, about 45%, about 50%, about 55%, about 60%, about 65%, about 70%, about 75%, about 80%, about 85%, about 90%, or ranges thereof). Alternatively, each component can comprise about 10% to 90% of the total hybrid weight or about 20% to about 80% of the total hybrid weight.

Unlike formulation approaches to improve drug performance by using inactive ingredients, the solid pharmaceutical hybrids of this invention improve drug performance by using two or more drug forms. The hybrids of this invention contain high concentrations of the forms of the agent. In particular, the solid pharmaceutical hybrids contain at least about 80% of their weight two or more forms of an active pharmaceutical agent, preferably, at least about 85%, and more preferably at least about 90% (e.g., at least about 91%, at least about 92%, at least about 93%, at least about 94%, at least about 95%, at least about 96%, at least about 97%, at least about 98%, or at least about 99%).

Since the solid pharmaceutical hybrid comprises two or more different drug forms, problems, such as a high crystallinity of a single form, are avoided. The solid pharmaceutical hybrid is superior to aforementioned formulation concepts where pharmacologically inactive molecules are introduced to drugs to improve drug properties, such as lactose to improve compressibility or a water-soluble adjuvant to improve wettability.

The composition comprising the solid pharmaceutical hybrid also can contain one or more carriers or excipients. The carriers or excipients must be acceptable in the sense of being compatible with the other ingredients and not deleterious to the recipient thereof. Examples of carriers or excipients for oral administration include cornstarch, lactose, magnesium stearate, talc, microcrystalline cellulose, stearic acid, povidone, crospovidone, dibasic calcium phosphate, sodium starch glycolate, hydroxypropyl cellulose (e.g., low substituted hydroxypropyl cellulose), hydroxypropylmethyl cellulose (e.g., hydroxypropylmethyl cellulose 2910), and sodium lauryl sulfate.

The solid pharmaceutical hybrid can be formulated into any suitable oral dosage form, such liquid and solid dosage forms. For example, the solid pharmaceutical hybrid can be formulated into suspensions, solutions, drops, syrups, two-piece hard shell capsules, soft gelatin capsules (softgel capsules), and tablets.

To formulate the softgel capsules, the solid pharmaceutical hybrid is dissolved in a soft capsule vehicle. Any suitable soft capsule vehicle can be used, such as polyethylene glycol or a mixture of polyethylene glycol and water. Polyethylene glycol having any suitable molecular weight can be used. Typically, the polyethylene glycol has a molecular weight of 300 to 1500, and preferably a molecular weight of 400 to 600. Typically, the water is purified when used in the soft capsule vehicle.

The softgel fills can be encapsulated into soft gelatin capsules using any suitable mechanism known in the art, such as rotary die technology (see J. P. Stanley, in The Theory and Practice of industrial Pharmacy; Lachman et al., Ed., Philadelphia, 1976).

The gelatin shell can be from any suitable source, such as bovine, porcine, fish, or poultry origin. The gelatin shell can be of any suitable bloom strength, such as about 100 to about 250 bloom (e.g., about 125 bloom, about 150 bloom, about 175 bloom, about 200 bloom, about 225 bloom) and preferably about 150 bloom.

The gelatin shell can be plasticized with tri- or poly-alcoholic plasticizers such as glycerin, sorbitol, xylitol, or mixtures thereof. A mixture of glycerol and sorbitol is preferred.

The softgel capsules can have any suitable moisture content, such as a total moisture content of less than 10% (e.g., less than about 9%, less than about 8%, less than about 7%, less than about 6%, less than about 5%, or ranges thereof) and preferably less than about 7%.

Hard shell capsules can be produced by filling the solid pharmaceutical hybrid of the invention into two piece capsules. The two piece capsules also can include lubricants and disintegrating agents as known in the art.

Liquid oral pharmaceuticals can be prepared by directly dissolving the solid pharmaceutical hybrid into a hydrophilic vehicle or a mixture of hydrophilic vehicles, such as water, propylene glycol, and glycerol.

Other adjuvants, such as sweeteners, flavor-enhancing agents, taste masking agents, anti-microbial preservatives, or viscosity imparting agents, can also be used in the compositions as known in the art.

Suitable sweeteners include, for example, saccharin sodium, sucrose, sorbitol, aspartame, and mannitol, or mixtures thereof.

Suitable flavoring agents include grape flavor, cherry flavor, cotton candy flavor, or other suitable flavor to make the liquid pharmaceutical easier for a patient to ingest. The flavoring agent or mixtures thereof are typically present in an amount of from about 0.0001 wt % to about 5 wt %.

Suitable anti-microbial preservatives include, for example, methylparaben, propylparaben, sodium benzoate, benzalkonium chloride, or mixtures thereof. The preservative or mixtures thereof are typically present in an amount of from about 0.0001 wt % to about 2 wt %.

Alternatively, the solid pharmaceutical hybrid can be used as powder for re-constitution with one or more adjuvants.

The composition comprising the solid pharmaceutical hybrid can be formulated into oral pharmaceuticals at any suitable dose. The dose depends on the particular agent for use in the hybrid. For example, the compositions can be formulated into solid dosage forms with agent dosages of 1 mg, 2 mg, 5 mg, 10 mg, 20 mg, 25 mg, 30 mg, 40 mg, 50 mg, 100 mg, 200 mg, 300 mg, 400 mg, 500 mg, 600 mg, 800 mg, 1000 mg, and ranges thereof.

The compositions also can be formulated into liquid suspensions or solutions at any suitable dose. Generally, the liquid concentrates can be formulated with agent concentrations of 1 mg/mL to 1000 mg/mL (e.g., 10 mg/mL, 20 mg/mL, 30 mg/mL, 40 mg/mL, 50 mg/mL, 60 mg/mL, 80 mg/mL, 100 mg/mL, 200 mg/mL, 300 mg/mL, 500 mg/mL, 550 mg/mL, 700 mg/mL, 800 mg/mL, and ranges thereof).

The following examples further illustrate the invention but, of course, should not be construed as in any way limiting its scope.

### EXAMPLE 1

This example demonstrates the preparation of a solid pharmaceutical hybrid comprising two or more forms of agent. In this example, the agent is ibuprofen and the hybrid comprises (a) a solid ibuprofen free acid and (b) a solid ibuprofen alkali salt generated by active mixing in presence of an aqueous medium.

A 2590 g batch of an ibuprofen solid pharmaceutical hybrid was prepared using the following ingredients.

| Ingredient | Amount (g) |
|---|---|
| Ibuprofen, USP | 2000 |
| Potassium bicarbonate | 120 |
| Potassium carbonate anhydrous, NF | 270 |
| Potassium hydroxide aqueous solution 33% (w/w) | 200 |

Ibuprofen was dry-mixed with potassium bicarbonate and potassium carbonate anhydrous in planetary (Hobart) mixer for 5 min. The blended composition was mixed with potassium hydroxide aqueous solution 33% (w/w) and additional water for 5 minutes and the wet hybrid was dried at 45° C.±5° C. in a drying oven. Dried ibuprofen hybrid was then passed through #40 mesh screen.

The resultant ibuprofen solid pharmaceutical hybrid comprised (a) a solid ibuprofen free acid and (b) a solid ibuprofen alkali salt generated by active mixing in presence of aqueous medium and had a final moisture content of 2% and a bulk density of 0.4 g/mL.

### EXAMPLE 2

This example demonstrates the preparation of an ibuprofen solid pharmaceutical hybrid comprising (a) a solid ibuprofen free acid and (b) a solid ibuprofen alkali salt generated by active mixing in presence of aqueous medium.

A 2470 g batch of an ibuprofen solid pharmaceutical hybrid was prepared using the following ingredients:

| Ingredient | Amount (g) |
|---|---|
| Ibuprofen, USP | 2000 |
| Potassium carbonate anhydrous, NF | 270 |
| Potassium hydroxide aqueous solution 33% (w/w) | 200 |

The ibuprofen solid pharmaceutical hybrid was prepared as set forth in Example 1.

The ibuprofen solid pharmaceutical hybrid comprised (a) a solid ibuprofen free acid and (b) a solid ibuprofen alkali salt generated by active mixing in presence of aqueous medium and had a final moisture content of about 2% and a bulk density of about 0.4 g/mL.

### EXAMPLE 3

This example demonstrates the preparation of a naproxen solid pharmaceutical hybrid comprising (a) a solid naproxen free acid generated by active mixing in presence of aqueous medium and (b) a solid naproxen alkali salt.

A 94.81 g batch of a naproxen solid pharmaceutical hybrid was prepared using the following ingredients.

| Ingredient | Amount (g) |
|---|---|
| Naproxen sodium, USP | 75.60 |
| Citric acid, USP | 19.21 |

Naproxen sodium was dry-mixed with citric acid in a planetary (Hobart) mixer for 5 min. The blend was moistened with purified water for 5 min and the wet mass was dried at 45°±5° C. in a drying oven. Dried naproxen solid hybrid was then screened through #30 mesh screen.

The resultant naproxen solid pharmaceutical hybrid comprised (a) a solid naproxen free acid generated by active mixing in presence of aqueous medium and (b) a solid naproxen alkali salt and had final moisture content of 2.2% and bulk density of 0.41 g/ml.

### EXAMPLE 4

This example demonstrates the preparation of a naproxen solid pharmaceutical hybrid comprising (a) a solid naproxen free acid generated by active mixing in presence of aqueous medium and (b) a solid naproxen alkali salt.

A 146 g batch of a naproxen solid pharmaceutical hybrid was prepared using the following ingredients.

| Ingredient | Amount (g) |
|---|---|
| Naproxen sodium, USP | 100.0 |
| Lactic acid (85%) | 18.18 |

Naproxen sodium was mixed with lactic acid solution for 5 min and the wet mass was dried at 45°±5° C. in a drying oven. The dried hybrid form of naproxen was then screened through #30 mesh screen.

The resultant naproxen solid pharmaceutical hybrid comprised (a) a solid naproxen free acid generated by active mixing in presence of an aqueous medium and (b) a solid naproxen alkali salt and 2.1% moisture content and a bulk density of 0.49 g/ml.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composition comprising a solid pharmaceutical hybrid,
wherein the solid pharmaceutical hybrid comprises two or more forms of an agent,
wherein the agent is a pharmacologically active agent, a therapeutic agent, or a diagnostic agent, and
wherein the solid pharmaceutical hybrid exhibits at least one superior property relative to one of the forms of the agent.

2. The composition of claim 1, wherein the superior property is selected from the group consisting of increased solubility, increased stability, increased bioavailability, reduced side effects, and increased activity.

3. The composition of claim 1, wherein the two or more forms of the agent are chemical forms of the agent.

4. The composition of claim 3, wherein the solid pharmaceutical hybrid comprises an ionizable form and non-ionizable form of the agent.

5. The composition of claim 3, wherein the solid pharmaceutical hybrid comprises at least two different polymorphs of the agent.

6. The composition of claim 3, wherein the solid pharmaceutical hybrid comprises at least two forms of the agent selected from the group consisting of the agent, a salt of the agent, an ester of the agent, a pro-drug of the agent, and an active metabolite of the agent.

7. The composition of claim 1, wherein the two or more forms of the agent are physical forms of the agent.

8. The composition of claim 7, wherein the solid pharmaceutical hybrid comprises at least one combination selected from the group consisting of:
(i) an anhydrous form and a hydrous form of the agent
(ii) an amorphous and a crystalline form of the agent;
(iii) at least two different particle size populations of the agent;
(iv) at least two crystalline forms of the agent;
(v) at least two different solvates of the agent;
(vi) at least two particles of differing geometric shapes;
(vii) at least two particles of differing bulk densities; and
(viii) at least two particles of differing flowability.

9. The composition of claim 8, wherein the solvates are hydrates.

10. A method of producing a solid pharmaceutical hybrid comprising two or more forms of an agent, comprising mixing a form of the agent with one or more different forms of the agent,
wherein the agent is a pharmacologically active agent, a therapeutic agent, or a diagnostic agent, and
wherein the solid pharmaceutical hybrid exhibits at least one superior property relative to one of the forms of the agent.

11. The method of claim 10, wherein the superior property is selected from the group consisting of increased solubility, increased stability, increased bioavailability, reduced side effects, and increased activity.

12. The method of claim 10, wherein the two or more forms of the agent are chemical forms of the agent.

13. The method of claim 12, wherein the solid pharmaceutical hybrid comprises an ionizable form and non-ionizable form of the agent.

14. The method of claim 12, wherein the solid pharmaceutical hybrid comprises at least two different polymorphs of the agent.

15. The method of claim 12, wherein the solid pharmaceutical hybrid comprises at least two forms of the agent selected from the group consisting of the agent, a salt of the agent, an ester of the agent, a pro-drug of the agent, and an active metabolite of the agent.

16. The method of claim 10, wherein the two or more forms of the agent are physical forms of the agent.

17. The method of claim 16, wherein the solid pharmaceutical hybrid comprises at least one combination selected from the group consisting of:
(i) an anhydrous form and a hydrous form of the agent
(ii) an amorphous and a crystalline form of the agent;
(iii) at least two different particle size populations of the agent;
(iv) at least two crystalline forms of the agent;
(v) at least two different solvates of the agent;
(vi) at least two particles of differing geometric shapes;
(vii) at least two particles of differing bulk densities; and
(viii) at least two particles of differing flowability.

18. The composition of claim 17, wherein the solvates are hydrates.

19. A method of producing a solid pharmaceutical hybrid comprising two or more forms of an agent, wherein the method comprises actively mixing one or more forms of an agent with one or more hybrid forming agents in a medium, such that the solid pharmaceutical hybrid is produced.

20. The method of claim 19, wherein the one or more hybrid forming agents are selected from the group consisting of physical energy and a chemical reagent.

21. The method of claim 19, wherein the medium is a fluid or solid medium.

22. The method of claim 19, wherein the agent is a pharmacologically active agent, a therapeutic agent, or a diagnostic agent, and wherein the solid pharmaceutical hybrid exhibits at least one superior property relative to one of the forms of the agent.

23. The method of claim 22, wherein the superior property is selected from the group consisting of increased solubility, increased stability, increased bioavailability, reduced side effects, and increased activity.

24. The method of claim 19, wherein the two or more forms of the agent are chemical forms of the agent.

25. The method of claim 19, wherein the two or more forms of the agent are physical forms of the agent.
